(19) United States
(12) Patent Application Publication
(10) Pub. No.: US 2003/0124680 A1
(43) Pub. Date: Jul. 3, 2003

(54) Alteration of acyltransferase domain substrate specificity

(76) Inventors: Christopher Reeves, Orinda, CA (US); Robert McDaniel, Palo Alto, CA (US); Ralph Reid, San Rafael, CA (US)

Correspondence Address: David P. Lentini, Kosan Biosciences, Inc., 3832 Bay Center Place, Hayward, CA, 94545, US

(21) Appl. No.: 10/214,796

(22) Filed: Aug. 7, 2002

Related U.S. Application Data
(60) Provisional application No. 60/310,730, filed on Aug. 7, 2001.

(51) Int. Cl. C12P 19/62; C07H 17/08; C12N 9/10; C12P 21/02; C12N 1/21; C12N 15/74

(52) U.S. Cl. 435/76; 435/69.1; 435/471; 435/252.3; 435/320.1; 435/193; 536/7.1

(57) ABSTRACT

Three distinct primary regions of acyltransferase (AT) domains of modular polyketide synthases (PKS) can be changed by site-specific mutagenesis of the corresponding coding sequences to change the specificity of the domain and the structure of the polyketide produced by the PKS.

### REFERENCE TO GOVERNMENT FUNDING

This invention was supported in part by SBIR grant DK57380. The U.S. government has certain rights in this invention.

### CROSS-REFERENCE TO RELATED APPLICATION

The present application claims the benefit of the filing date of provisional U.S. patent application Serial No. 60/310,730, filed Aug. 7, 2001, incorporated herein by reference.

### FIELD OF THE INVENTION

The present invention provides methods for altering the subunit specificity of acyltransferase domains of polyketide synthase (PKS) enzymes, as well as PKS altered by the method, recombinant vectors encoding them, host cells expressing them, and the polyketides so produced. The present invention relates to the fields of molecular biology, biochemistry, medicinal chemistry, and medicine.

### BACKGROUND OF THE INVENTION

Polyketides are a structurally diverse class of natural products that have played an important role in the development of therapeutic and agricultural chemicals (1, 2—all references cited herein are incorporated herein by reference and indicated by a number corresponding to the numbered location in the list of references cited at the conclusion of the “Detailed Description of the Invention”, below). Many polyketides possess antibiotic, anticancer, immunosuppresive or other useful biological properties. In recent years, researchers have developed a number of protein engineering strategies to manipulate polyketide synthases (PKSs), the enzyme complexes responsible for biosynthesis of polyketides, in order to generate novel chemical structures (3, 4). PKSs assemble the carbon backbones of polyketides through repeated condensations between acyl-CoA thioesters, typically malonyl, methylmalonyl and ethylmalonyl. Thus, the diversity observed in polyketide structures is in part derived from the incorporation of different starter or extender monomers, which is controlled by the PKS.

In modular PKS systems, such as the 6-deoxyerythronolide B synthase (DEBS), selection of the extender unit for each module, as well as the starter unit in many cases, is carried out by an acyltransferase (AT) domain. An AT domain catalyzes the transacylation of the monomer unit from CoA to the phosphopantethiene arm of the acyl carrier protein (ACP) in the same module. AT domains generally possess a stringent specificity for a single acyl-CoA substrate in their natural context, although some ATs can incorporate at least two different monomers with similar efficiency. Alignments between methylmalonyl-CoA (mmCoA) specific and malonyl-CoA (mCoA) specific AT domain sequences always cluster into two groups according to the specificity of the domain (5-7). Haydock et al. have identified two possible divergent sequence motifs based on such alignments and comparison to the Escherichia coli malonyl-CoA:ACP transacylase (FabD) crystal structure (8, 9) but have not demonstrated the contribution of these motifs to substrate specificity experimentally. In a separate study, Lau et al. used in vivo experiments to locate a different variable region at the C-terminal segment of AT domains that may influence specificity (10). Because this segment of the AT shows little homology between different AT domains, it was not clear how these amino acids contributed mechanistically to substrate selectivity.

Despite the elucidation of potential regions within ATs for engineering specificity, the most popular method of changing substrate utilization in PKSs has been via cassette mutagenesis of AT domains in which the entire ˜300-350 amino acid domain of one module is exchanged with a homologous AT cassette that encodes a different starter or extender unit from another, usually heterologous, PKS module. This technique has proven to be generally robust. A number of successful AT replacements with modular PKSs have been reported, in each case producing a new polyketide with the predicted change (11-16). However, occasionally AT replacements are only marginally successful or entirely unsuccessful, leading to only very small amounts of the desired compound or no product at all (14). For example, an AT cassette from module 2 of the rapamycin PKS was used successfully to alter the extender unit specificity from methylmalonate to malonate in modules 1, 2, 3, 5, and 6 of DEBS (16). However, this same AT cassette failed to produce a polyketide at detectable levels under the conditions tested when it was introduced in module 4 of DEBS.

Assuming that the genetic engineering of the PKS is properly executed, there are two possible reasons for such low production. Either the foreign AT domain causes the PKS complex to fold incorrectly and lose, in whole or in part, a necessary activity, or the replacement leads to a modified polyketide chain that is not or only poorly recognized as a substrate by a subsequent (“downstream”) PKS activity. In the case of the former, it might be possible to overcome this liability if the AT domain specificity was engineered in a manner that minimized perturbation to the tertiary structure of the module—e.g. mutagenesis to a limited number of amino acids. To do so, however, requires an understanding of the rules for proper PKS folding and the structural features involved in substrate recognition. Thus, there remains a need for methods to alter the domain specificity of an AT domain of a modular PKS such that the resulting PKS can produce a polyketide, and there remains a need for polyketides produced by the method, including but not limited to the polyketide 6-desmethyl-6-deoxyerythronolide B and the erythromycin and motilide compounds corresponding thereto.

### SUMMARY OF THE INVENTION

The present invention provides a method for the altering the AT specificity of a module of a PKS by changing a limited number of the amino acid residues in the domain. In one embodiment, site-specific mutagenesis is used to make the changes in the coding sequence of a nucleic acid that encodes the AT domain. In another embodiment, the AT domain is from extender module 4 of DEBS.

The present invention also provides recombinant PKS enzymes comprising one or more AT domains altered by the method, as well as vectors encoding such enzymes, and host cells comprising such vectors.

The present invention also provides polyketides produced by the host cells of the invention. In one embodiment, the polyketide is 6-desmethyl-6-deoxyerythronolide B, a glycosylated or hydroxylated derivative thereof, and corresponding erythromycins thereof.

In another embodiment the erythromycins corresponding to 6-desmethyl-6-deoxyerythronolide B are produced by providing the compound to a culture of Saccharopolyspora erythraea under conditions such that the corresponding erythromycins are produced. In another embodiment, the erythromycins corresponding to 6-desmethyl-6-deoxyerythronolide B are produced by expressing the DEBS genes of the present invention in Saccharopolyspora erythraea and culturing the host cell comprising those genes under conditions such that the genes are expressed and the corresponding erythromycins are produced.

These and other embodiments, modes, and aspects of the invention are described in more detail in the following description, the examples, and claims set forth below.

### DETAILED DESCRIPTION OF THE INVENTION

Utilizing the crystal structure of E. coli FabD (9) and amino acid alignments of different modular PKS AT domains, key residues predicted to contribute to the specificity of AT domains were identified, and based on these predictions, three independent site-directed mutant ATs in module 4 of DEBS were constructed. Two of the regions engineered corresponded to those previously identified by Haydock et al. as important for specificity (8), whereas the third did not. All of the mutants permitted the incorporation of a malonate (as well as a methylmalonate) unit at the corresponding position of the polyketide, thereby producing 6-desmethyl-6-deoxyerythronolide B (6-desmethyl-6-dEB) for the first time. A series of cassette substitutions in module 4 using malonyl-CoA specific AT domains from four different heterologous sources as well as a hybrid module 4, however, did not produce any polyketide. Together, these results suggest that module 4 of DEBS is particularly sensitive to heterologous domain replacement and that the methods of the present invention are useful alternative methods for engineering specificity of modular PKSs.

All six of the extender AT domains in DEBS are specific for 2(S)-methylmalonyl-CoA (21). Five different 6-deoxyerythronolide B (6-dEB) analogs have been produced by replacing the AT domains from modules 1, 2, 3, 5, or 6 of DEBS with the AT domain from module 2 of the rapamycin PKS (rapAT2) (16). Production titers of the corresponding 12-, 10-, 8-, 4-, and 2-desmethyl-6dEB analogs ranged from ˜4-70% of the amount of 6-dEB produced by unmodified DEBS. This same cassette was also used to replace the AT domain from module 4 of DEBS (AT4) as described in the Examples below. Expression of this PKS in S. lividans did not lead to any detectable levels of the desired polyketide 6-desmethyl-6-dEB. This result was somewhat surprising, because the cassette functioned in every other DEBS module.

To determine if the problem resulted from an incompatibility between the rapAT2 domain and DEBS module 4, three additional AT swaps were made using ATs from the FK520 PKS and the epothilone PKS (EpoPKS). Each of the chosen ATs, fk520AT10, epoAT3 and epoAT4, posses a greater sequence similarity to DEBS AT4 than does rapAT2. The first two AT domains incorporate only malonate extender units in their native PKSs whereas the epoAT4 domain appears to be capable of incorporating either malonate or methylmalonate in epothilone biosynthesis (18). Therefore, a functional substitution with this domain should result in production of both 6-dEB and 6-desmethyl-6dEB in the presence of both precursors. An alternative set of junctions between the native and heterologous sequences was also used in these replacements to reduce the amount of heterologous sequence introduced, while still retaining those regions believed to be important for specificity. The new sites were 43 amino acids downstream and 60 amino acids upstream of the right and left junctions, respectively, used in the substitution with the rapAT2 cassette. Again, however, the expected 6-desmethyl compound could not be detected when the recombinant PKSs were expressed in S. lividans. Furthermore, 6-dEB, the natural product of DEBS, was also absent from the epoAT4 swap.

Another concern for the apparent inability of malonyl-CoA specific ATs to function in module 4 of DEBS was the specificity of the downstream enzymatic domains which reduce the beta-ketone to a methylene after condensation with the extender unit. Module 4 contains ketoreductase (KR), dehydratase (DH) and enoylreductase (ER) domains of which one or more could possess severely attenuated activity for the alpha-desmethyl pentaketide intermediate. A hybrid module containing the KS from DEBS module 4 and the AT, DH, ER, KR, and ACP domains from the epothilone PKS module 5 was constructed. The EpoPKS module 5 incorporates a malonate extender unit and so the reductive domains from this module process a polyketide intermediate with the appropriate alpha-carbon functionality. This PKS also did not produce any compound related to 6-dEB that could be detected.

Because neither of the above approaches afforded the desired 6-desmethyl analog, the structurally more conservative methods of the present invention were developed and used to generate the compound. The E. coli FabD crystal structure was studied to locate regions in or near the active site with amino acid sequences showing a strong correlation between malonyl and methylmalonyl specificity in AT alignments. Three such regions were identified and targeted for mutagenesis of DEBS AT4; in each case, the region was mutated from the canonical mmCoA-specific motif to the canonical mCoA-specific motif using site-directed mutagenesis, as described below. The method can be employed in reverse to change a mCoA-specific motif to a mmCoA-specific motif. Region 1, immediately upstream of the highly conserved Gln residue in the active site (Gln-63 in FabD), and region 2, a single amino acid adjacent to the active site serine residue (Ser-92 in FabD) have been implicated in specificity of AT domains based on sequence alignments (8) but have never been directly proven to play a role in specificity. Region 3 is adjacent to a highly conserved histidine residue (His-201) that lies near the active site serine in three dimensional space.

These regions are shown below utilizing the DNA and amino acid sequences corresponding to the DEBS module 4 AT.

Region 1—in DEBS module 4 AT, specific for methylmalonyl CoA:

6229 6249
GAG CGC GTC GAC GTG CTC CAG
E R V D V L Q

Region 1 in mutated DEBS module 4 AT of the invention, able to bind malonyl and methylmalonyl CoA:

6229 6249
GAG GAC GTC CTC TAC GCC CAG
E D V L Y A Q

Mutations to provide the amino acid sequences EDDLYAQ and EDTLYAQ are also preferred for Region 1.

Region 2—in DEBS module 4 AT, specific for methylmalonyl CoA:

6325 6345
GGC CAC TCG CAG GGC GAG ATC
G H S Q G E I

Region 2—in mutated DEBS module 4 AT of the invention, able to bind malonyl and methylmalonyl CoA:

6325 6345
GGC CAC TCG CTC GGC GAG ATC
G H S L G E I

Mutations to provide the amino acid sequences GHSVGEI and GHSIGEI are also preferred for Region 2.

Region 3—in DEBS module 4 AT, specific for methylmalonyl CoA:

6625 6645
GTG CGC TAC GCC TCC CAC TCC
V R Y A S H S

Region 3—in mutated DEBS module 4 AT of the invention, able to bind malonyl and methylmalonyl CoA:

6625 6645
GTG CGC CAC GCC TTC CAC TCC
V R H A F H S

Mutation to provide the amino acid sequence VRHGFHS is also preferred for Region 3.

All three of the mutant PKSs yielded similar product profiles when expressed in S. lividans. Based on LC/MS analysis, each PKS produced a combination of 6-dEB and two additional compounds, each whose mass was 14 amu smaller than 6-dEB. One of these displayed the same retention and mass fragmentation as 8,8a-deoxyoleandolide (8,8a-dOle). This compound is formed by priming of the DEBS loading domain with acetate rather than propionate and is usually observed as a minor component when DEBS is expressed in heterologous hosts (22). The other compound was presumed to be the desired 6-desmethyl-6dEB analog and was purified for structural characterization. ¹H-NMR and ¹³C-NMR spectroscopy established definitively that this compound was 6-desmethyl-6-dEB. Thus, in each of the PKSs, the specificity of the AT domain has been altered to incorporate a malonate extender unit, although not exclusively. The total yields of polyketides were ˜5-10 mg/L under the conditions used. The ratios of the polyketide products for each PKS were slightly different, with the region 3 mutations yielding the highest ratio of mCoA to mmCoA incorporation.

There have been several examples of mmCoA to mCoA AT replacements reported that collectively include every module of DEBS except module 4 (12-14, 16). Stassi et al. were able to make a functional AT swap in module 4 with an ethylmalonyl-CoA specific AT which was capable of incorporating either a butyryl unit in the presence of ethylmalonyl-CoA or a propionate unit if no ethylmalonyl-CoA was available (15). However, a substantial decrease in polyketide titer is believed to have accompanied the substitution. As noted above and in the Examples, the substitution of DEBS AT4 with four different heterologous mCoA AT cassettes did not yield any measurable polyketide production under the test conditions employed. The reason for the apparent low or non-production from these substitutions is not known. However, the subsequent synthesis of 6-desmethyl-6-dEB by the site-specific AT4 mutations in reasonable yield demonstrates that DEBS can process the unnatural pentaketide chain lacking an alpha-methyl. Together, these results suggest that substitution of AT domains in module 4 might alter the conformation of the module so as to disrupt efficient transfer of the acyl-CoA substrate to the ACP domain.

This hypothesis is best supported by the substitution with the epoAT4 cassette, which should not pose any substrate barriers to DEBS, because of its presumed relaxed mCoA and mmCoA specificity. Furthermore, some previously reported AT substitutions have resulted in a ‘relaxation’ of specificity, which suggests that the rest of the module can affect the conformation of a heterologous AT domain (15, 23). This hypothesis is also consistent with the observation that alterations to a region at the C-terminus of AT domains, and located at a considerable distance from the active site, can still affect substrate specificity (10), possibly through steric forces that distort the AT domain.

The E. coli FabD crystal structure shows five water molecules in a plane just above and surrounding the active site serine (Ser-92). Five atoms of an acyl-CoA substrate—the thioester carbonyl oxygen and sulfur atoms, the two carboxylate oxygen atoms, and the alpha carbon substituent atom—can be positioned in the active site such that they occupy nearly the same positions as these water molecules.

The geometry of substrates in the active site of AT domain can be hypothesized to exist as follows. The cleft containing the active site is oriented vertically and lies in a plane slightly above the catalytic serine (Ser-92 in E. coli FabD). The rest of the CoA (or ACP) moiety rises up out of the active site cleft. This configuration places the carboxyl group of the substrate in proximity to a conserved arginine residue (Arg-117 in FabD) and is therefore consistent with biochemical experiments that have directly implicated this residue in carboxylate binding (24). In this model, the substituent on the alpha-carbon of an acyl-CoA substrate would point toward a hydrophobic pocket with a conserved Gln residue (equivalent to Gln-11 of E. coli FabD) lying immediately above it.

The following table shows an alignment of divergent motifs in PKS AT domains. The divergent sequence blocks at or near the active site of PKS AT domains that have a correlation with malonyl versus methylmalonyl specificity are shown. The left block is about 30 residues toward the N-terminus from the active site Ser in the middle block. The right block ends with the invariant His residue (His-201 in E. coli FabD) found in the active site. The residue just following the invariant GHS motif is a branched chain amino acid in all known malonyl-specific AT domains or a glutamine in most methylmalonyl-specific AT domains. It is part of a hydrophobic pocket that may be involved in recognition of the different malonyl-CoA substrates. Abbreviations are epo, epothilone; sor, soraphen; asc, ascomycin; rap, rapamycin; nid, niddamycin. In the few cases where AT domains of the same specificity from the same cluster had different sequences within a block, the consensus sequence is shown.

TABLE 1
Alignment of Divergent Motifs in PKS AT Domains

| E. coli | 63 | 92 | 201 |
|---|---|---|---|
| FabD residue # | \| | \| | \| |
| epo malonyl | TAFTQ | GHSIGE | HAFH |
| sor malonyl | TAFTQ | GHSIGE | HAFH |
| 520 malonyl | TLYAQ | GHSIGE | HAFH |
| rap malonyl | TGYAQ | GHSVGE | HAFH |
| nid malonyl | TEYTQ | GHSVGE | HAFH |
| nid methyl | VDVVQ | GHSQGE | YASH |
| sor methyl | VDVVQ | GHSQGE | YASH |
| epo methyl | IDVVQ | GHSMGE | VASH |
| 520 methyl | VEVVQ | GHSQGE | YASH |
| rap methyl | VDVVQ | GHSQGE | YASH |
| epo relaxed | TAFTQ | GHSAGE | HASH |

In accordance with the methods of the present invention, three different motifs within the DEBS AT 4 domain can be engineered to change the substrate selectivity from mCoA exclusively to allow both mCoA and mmCoA. Because the intracellular concentrations of these two substrates are not known in S. lividans, it is not possible to calculate the relative specificities of the mutant AT domains based on the polyketide titers. These could be addressed and calculated with in-vitro systems such as those recently developed for individual modules of DEBS (25, 26).

Based on the E. coli FabD structure, all three of these regions lie within the active site cleft of the AT domain. Prior to the present invention, the divergent motifs of regions 1 and 2 could be used to predict the specificity of a mCoA or mmCoA AT domain, but it was not known if these motifs were directly involved in substrate selectivity or were only an indication of specificity resulting from evolutionary constraints. Furthermore, the methods of the invention can be used to target a third isolated region of AT domain generally. This third region has an even greater influence on specificity than the other two in the DEBS AT4 domain.

The consensus motifs for regions 1 and 2 are the same for both mmCoA ATs as well as ethylmalonyl (em) AT domains; therefore, how these amino acids would contribute directly to binding of the two different substrates is not apparent. However, the consensus motifs for region 3 are different for mCoA (where the motif is His-X-Phe-His, typically His-Ala-Phe-His), mmCoA ATs (Tyr-X-Ser-His or Val-X-Ser-His, where X is typically Ala), emCoA ATs (Thr-Ala-Gly-His or Thr-Ala-Ser-His or Cys-Pro-Thr-His), and methoxymalonyl (omem) CoA (Phe-Ala-Gly-His or Val-Ala-Gly-His). In the preceding motifs, X is independently selected from any amino acid. In particular embodiments, the mutations introduced in region 3 to provide an omemCoA specific AT domain are selected based on the sequence of the starting domain. Thus, if the AT domain to be mutated is a malonyl CoA specific domain having a region 3 motif HAFH, that sequence is changed to HAGH to yield an omemCoA specific domain. If the AT domain to be mutated is a methylmalonyl CoA specific domain having a region 3 motif YASH, that sequence is changed to FASH to yield an omemCoA specific domain. If the AT domain to be mutated is an ethylmalonyl CoA specific domain having a region 3 motif TAGH, that sequence is changed to FAGH to yield an omemCoA specific domain.

The methods of the invention thus allow one to change the specificity of one type of AT to another by targeting region 3 for mutagenesis as exemplified herein. The size differences in side chains between the Phe, Ser, or Gly residues immediately adjacent to the conserved His residue (His-201) could possibly account for the differences in the corresponding acyl side chains of mCoA, mmCoA, emCoA, and omemCoA substrates. Regardless, the methods of the present invention allow alterations of the amino acid sequences in region 3 to change one motif to another and correspondingly to change the specificity of the AT domain so changed.

It is surprising that all three regions can be altered individually to change the specificity of the domain. This result indicates that many different amino acids can contribute to substrate binding, perhaps either directly or indirectly. Therefore, it is likely that the degree to which the mutations described here will affect AT specificity will vary among different AT domains and PKSs. Alteration of two or all three of these regions could improve productivity and specificity and is within the scope of the present invention. High throughput screening techniques of isolated AT domains or modules could be used to find mutants with even better properties than those described here. Furthermore, the identification of these critical residues for substrate utilization could facilitate the tailoring of AT specificity for non-natural extender units and used in conjunction with recent precursor engineering techniques (27) to enhance the diversity of ketide units that can be used in combinatorial biosynthesis technologies.

Other features and advantages of the invention will be apparent from the following detailed description, and from the claims.

The following references are referred to herein by the number shown to the left of each reference citation; each of the references cited is incorporated herein by reference.

1. O'Hagan, D. (1991) The polyketide metabolites, Ellis Horwood, Chichester, UK.

2. Hopwood, D. (1997) Chem. Rev. 97, 2465-2497.

3. McDaniel, R., and Khosla, C. (2001) in Enzyme technologies for pharmaceutical and biotechnological applications (Kirst, H. A., Yeh, W.-K., and Zmijewski Jr., M. J., Eds.) pp 397-426, Marcel Dekker, Inc., New York.

4. Weissman, K. J., and Staunton, J. (2001) in Enzyme technologies for pharmaceutical and biotechnological applications (Kirst, H. A., Yeh, W.-K., and Zmijewski Jr., M. J., Eds.) pp 427-470, Marcel Dekker, Inc., New York.

0.5. Schwecke, T., Aparicio, J. F., Molnar, I., Konig, A., Khaw, L. E., Haydock, S. F., Oliynyk, M., Caffrey, P., Cortes, J., Lester, J. B., Böhm, G. A., Staunton, J., and Leadlay, P. F. (1995) Proc. Natl. Acad. Sci., U.S.A. 92, 7839-7843.

6. Kakavas, S. J., Katz, L., and Stassi, D. (1997) J. Bacteriol. 179, 7515-7522.

7. Wu, K., Chung, L., Revill, P. W., Katz, L., and Reeves, C. D. (2000) Gene 251, 81-90.

8. Haydock, S. F., Aparicio, J. F., Molnár, I., Schwecke, T., Khaw, L. E., König, A., Marsden, A. F. A., Galloway, I. S., Stuanton, J., and Leadlay, P. F. (1995) FEBS Lett. 374, 246-248.

9. Serre, L., Verbree, E. C., Dauter, Z., Stuitje, A. R., and Derewenda, Z. S. (1995) J. Biol. Chem. 270, 12961-12964.

10. Lau, J., Fu, H., Cane, D. E., and Khosla, C. (1999) Biochem. 38, 1643-1651.

11. Kuhstoss, S., Huber, M., Turner, J. R., Paschal, J. W., and Rao, R. N. (1996) Gene 183, 231-236.

12. Oliynyk, M., Brown, M. J. B., Cortes, J., Staunton, J., and Leadlay, P. F. (1996) Chem. & Biol. 3, 833-839.

13. Liu, L., Thamchaipenet, A., Fu, H., Betlach, M., and Ashley, G. (1997) J. Am. Chem. Soc. 119, 10553-10554.

14. Ruan, X. R., Pereda, A., Stassi, D. L., Zeidner, D., Summers, R. G., Jackson, M., Shivakumar, A., Kakavas, S., Staver, M. J., Donadio, S., and Katz, L. (1997) J. Bacteriol. 179, 6416-6425.

15. Stassi, D. L., Kakavas, S. J., Reynolds, K. A., Gunawardana, G., Swanson, S., Zeidner, D., Jackson, M., Liu, H., Buko, A., and Katz, L. (1998) Proc. Natl. Acad. Sci. USA 95, 7305-7309.

16. McDaniel, R., Thamchaipenet, A., Gustafsson, C., Fu, H., Betlach, M., Betlach, M., and Ashley, G. (1999) Proc. Natl. Acad. Sci. USA 96, 1846-1851.

17. Tang, L., and McDaniel, R. PCT Patent Publication No. 00/026349; see also PCT Patent Publication Nos. 01/27284 and 01/83803.

18. Tang, L., Shah, S., Chung, L., Carney, J., Katz, L., Khosla, C., and Julien, B. (2000) Science 287, 640-642.

19. Molnár, I., Schupp, T., Ono, M., Zirkle, R. E., Milnamow, M., Nowak-Thompson, B., Engel, N., Toupet, C., Stratman, A., Cyr, D. D., Gorlach, J., Mayo, J. M., Hu, A., Goff, S., Schmid, J., and Ligon, J. M. (2000) Chem. Biol. 7, 97-109.

20. Kao, C. M., Luo, G., Katz, L., Cane, D. E., and Khosla, C. (1995) J. Am. Chem. Soc. 117,9105-9106.

21. Marsden, A. F. A., Caffrey, P., Aparicio, J. F., Loughran, M. S., Staunton, J., and Leadlay, P. F. (1994) Science 263, 378-380.

22. Kao, C. M., Katz, L., and Khosla, C. (1994) Science 265, 509-512.

23. Xue, Q., Ashley, G., Hutchinson, C. R., and Santi, D. V. (1999) Proc. Natl. Acad. Sci. USA 96, 11740-11745.

24. Rangan, V. S., and Smith, S. (1997) J. Biol. Chem. 272, 11975-11978.

25. Gokhale, R. S., Tsuji, S. Y., Cane, D. E., and Khosla, C. (1999) Science 284, 482-485.

26. Wu, N., Kudo, F., E., C. D., and Khosla, C. (2000) J. Am. Chem. Soc. 122, 4847-4852.

27. Pohl, N. L., Hans, M., Lee, H. Y., Kim, Y. S., Cane, D. E., and Khosla, C. (2001) J. Am. Chem. Soc. 123, 5822-5823.

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

### EXAMPLES

### Example 1

### Strains, Culture Conditions, and DNA Manipulation

All strains, culture conditions and DNA manipulations were essentially as described (17) unless noted otherwise. In the construction of the final S. lividans strains used for expression of polyketides, a second plasmid, pBOOST, was included. This plasmid, in conjunction with the pRM5-based expression plasmids used here increases polyketide production by a mechanism that is currently under investigation (see U.S. patent application Serial Nos. 10/126,196, filed Apr. 19, 2002, and No. 60/259,289, filed Dec. 28, 2000, both of which are incorporated herein by reference).

### Example 2

### Construction of AT Replacements in DEBS Module 4

Four different AT cassettes were used for substitution of the AT domain in module 4. The rapAT2 cassette has been described (16). The remaining three cassettes—FK520AT10, epoAT3, and epoAT4—were PCR amplified from DNA encoding the FK520 (7) and epothilone PKSs (18, 19) using oligonucleotide primers. For replacement of the rapAT2 cassette, BamH I and Pst I restriction sites were engineered in positions flanking the AT4 domain which correspond to the same positions used in other modules of DEBS with the same cassette through a series of subcloning and PCR steps using standard techniques.

To replace DEBS AT4 with the other three cassettes (as well as the AT4 site-specific mutants discussed below), a different set of restriction sites was engineered within the AT4 domain. A subclone was generated in which unique Hind III and Pst I sites were introduced in AT4 using PCR amplification of segments ˜1 kb upstream of the Hind III site, ˜1 kb downstream of the Pst I site and between the two sites. The three PCR products were assembled to give pKOS131-62A, which was verified by DNA sequencing. Both restriction sites are silent, and the altered sequences are as follows (restriction sites underlined, altered nucleotides in bold):

5′-CTCTCGCCGCACACCGACTGGAAGCTTCTCGACGTCGTCCGCGGCGA

CGGC;

5′-CTGCTGATGGCGGTCGAGGAGACTGCAGAGCACGCGGGCGCGGAAGT

CACC.

The final expression plasmids were constructed as follows. The vector pKOS11-77 contains the DEBS genes with an engineered Spe I site downstream of the ACP of module 2 (20). This site appears to be functionally silent based on production of 6-dEB by pKOS11-77 in S. lividans (16). The Spe I to Bgl II fragment containing the gene for DEBS2 was subcloned from pKOS11-77 into Litmus 28 (New England Biolabs) and the Hind III and Pst I sites were removed from the polylinker by cutting with Hind III and Xba I and inserting a linker to give pKOS131-68. The BsiW I to BamH I fragment was subcloned from pKOS11-77 into Litmus 28 to give pKOS131-57. The Apa I to Not I fragment of pKOS131-62A was moved into pKOS131-57 to give pKOS131-72B and the BsiW I to Fse I fragment from pKOS131-72B was moved into pKOS131-68 to give pKOS131-72A. Each of the three heterologous AT domains (FK520AT10, epoAT3 and epoAT4) were moved as Hind III-Pst I fragments into pKOS131-72A. At this stage the AT inserts of each subclone were confirmed by DNA sequencing. The Spe I to Bgl II fragment from each of the three subclones was moved into pKOS11-77 to obtain the final PKS expression vectors.

### Example 3

### Mutagenesis of DEBS AT4

Site-directed mutations were introduced in three regions of DEBS AT4 using the Altered-Sites mutagenesis kit (Promega). The Hind III-Pst I PCR product from AT4 was cloned into pAlter, and the mutations described for regions 1, 2, and 3 above were introduced following protocols provided by the manufacturer. All mutations were verified by DNA sequencing. Each of the mutant cassettes was introduced into the Hind III and PstI sites of pKOS131-72A. Final expression plasmids were made analogous to the above AT substitutions.

### Example 4

### Production and Analysis of Polyketides

Each of the expression plasmids was introduced in S. lividans K4-114 also containing pBOOST. Supernatants of strains grown in R5 liquid (+50 mg/L thiostrepton and 200 mg/L apramycin) were analyzed using an LC-MS system equipped with on-line extraction. The LC-MS system was comprised of a 10 port, 2 position switching valve/injector, Beckman System Gold HPLC, an Alltech ELSD detector, and a PE SCIEX API100 LC MS-based detector equipped with an atmospheric pressure chemical ionization source. For 6-dEB and 6-desmethyl-6-DEB, 100 microliters of clarified whole broth was loaded onto the guard column after a 1 min pre-equilibration with H₂O at 1 ml/min. At 30 sec post-injection, a linear gradient to 15% MeCN over 1 min was initiated. At 2 min the direction of flow through the guard column was reversed, and the eluent was diverted onto a Metachem Inertsil ODS-3 column (5 □m, 4.6×150 mm) pre-equilibrated with 15% MeCN. A linear gradient from 15 to 100% MeCN at 1 ml/min over 6 min, then 100% MeCN for 3 min was monitored by ELSD and MS. Authentic purified 6-dEB and 8,8a-dOle were used as a controls for compound verification. 6-dEB was used to generate a calibration curve for polyketide titer measured by ELSD.

### Example 5

### Purification and Characterization of 6-Desmethyl-6-dEB

A host cell of the invention that produces 6-desmethyl-6-dEB, S. lividans K4-114/pKOS164-35G+pBOOST, was grown in 2L of R5 as above. The cells were removed by centrifugation, and the broth was filtered through a 2.5×30 cm column of XAD-16 adsorbent resin. The resin was washed with two volumes of water and then eluted with four volumes of acetone. The acetone eluate was evaporated on a rotary evaporator to a dark aqueous slurry, which was extracted three times with equal volumes of ethyl acetate. The organic extracts were combined, washed sequentially with saturated aqueous NaHCO₃ and brine, then dried over MgSO₄, filtered, and evaporated. The oily yellow residue was dissolved in 2 ml of CH₃CN, diluted with 2 ml of water, and filtered through a C₁₈ Mega Bond-Elut (Varian) extraction column. The column was washed with 10 ml of 1:1 CH₃CN/H₂O, and the total eluate was evaporated. Final purification was by preparative HPLC, using a 20×50 mm C₁₈ InertSil (Metachem) column and a gradient from 20% to 100% CH₃CN in water at a flow rate of 10 ml/min. Detection was by UV absorbance at 210 n, with peak identity confirmed by mass spectrometric analysis. The appropriate HPLC fraction was evaporated, yielding 2 mg of 6-desmethyl-6-dEB. ¹H-NMR (CDCl₃, 400 MHz): δ 5.154 (1H, ddd, J=1.2, 4.0, 9.6 Hz, H-13); 4.038 (1H, br d, J=10.4 Hz, H-5); 3.984 (1H, br d, J=10.8 Hz, H-3); 3.779 (1H, dd, J=0.8, 4.8 Hz, OH-11); 3.710 (1H, ddd, J=2.4, 4.8, 10.4 Hz, H-11); 3.021 (1H, d, J=2.4 Hz, OH-3); 2.803 (1H, dq, J=6.8, 10.8 Hz, H-2); 2.698 (1H, br q, J=6.8 Hz, H-10); 2.53 (1H, m, H-8); 2.186 (1H, br s, OH-5); 1.82 (unres., H-14a); 1.80 (unres., H-6a); 1.80 (unres., H-4); 1.74 (unres., H-7a); 1.73 (unres., H-12); 1.65 (1H, m, H-6b); 1.53 (1H, m, H-14b); 1.318 (3H, d, J=6.8 Hz, Me-2); 1.260 (1H, m, H-7b); 1.051 (3H, d, J=6.4 Hz, Me-8); 1.022 (3H, d, J=6.8 Hz, Me-10); 1.011 (3H, d, J=6.8 Hz, Me-4); 0.937 (3H, t, J=7.4 Hz, H-15); 0.882 (3H, d, J=6.8 Hz, Me-12). ¹³C-NMR (CDCl₃, 100 MHz): δ 213.02 (C-9), 178.52 (C-1), 78.86 (C-3), 76.10 (C-13), 74.21 (C-5), 71.01 (C-11), 44.63 (C-2), 44.07 (C-10), 40.71 (C-12), 38.73 (C-8), 37.01 (C-4), 29.76 (C-6), 28.58 (C-7), 25.46 (C-14), 14.80 (Me-2), 12.28 (Me-8), 10.63 (C-15), 9.22 (Me-12), 6.05 (Me-10), 3.28 (Me-4). Unresolved ¹H resonances were assigned from two-dimensional correlation experiments.

Numerous modifications may be made to the foregoing systems without departing from the basic teachings thereof. Although the present invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow. All publications or patent documents cited in this specification are incorporated herein by reference as if each such publication or document was specifically and individually indicated to be incorporated herein by reference.

Citation of the above publications or documents is not intended as an admission that any of the foregoing is pertinent prior art, nor does it constitute any admission as to the contents or date of these publications or documents.

What is claimed is:

1. A method for altering substrate specificity of an acyltransferase domain of a modular polyketide synthase, said method comprising changing one or more amino acid residues in one or more regions of said domain, said regions selected from one or more of the group consisting of (i) region 1, immediately upstream of a highly conserved Gln residue in an active site; (ii) region 2, a single amino acid adjacent to an active site serine residue; and (iii) region 3, adjacent to a highly conserved histidine residue that lies near the active site serine in three dimensional space.

2. The method of claim 1, wherein said substrate specificity is changed from methylmalonyl to malonyl.

3. A polyketide that is 6-Desmethyl-6-deoxyerythronolide B.

4. A hydroxylated and/or glycosylated derivative of the compound of claim 3.

5. The derivative of claim 4 that is 6-desmethyl-erythromycin A.

6. The derivative of claim 4 that is 6-desmethyl-erythromycin B.

7. The derivative of claim 4 that is 6-desmethyl-erythromycin C.

8. The derivative of claim 4 that is 6-desmethyl-erythromycin D.

9. A host cell that comprises a polyketide synthase (PKS), said PKS comprising an acyltransferase (AT) domain produced from a coding sequence that has been generated by site-specific mutagenesis of a naturally occurring coding sequence to change one or more amino acid residues in one or more regions of said domain, said regions selected from one or more of the group consisting of (i) region 1, immediately upstream of a highly conserved Gln residue in an active site; (ii) region 2, a single amino acid adjacent to an active site serine residue; and (iii) region 3, adjacent to a highly conserved histidine residue that lies near the active site serine in three dimensional space.

10. The method of claim 2, wherein said region is region 1, and an amino acid sequence ERVDVLQ is changed to EDVLYAQ, EDDLYAQ, or EDTLYAQ.

11. The method of claim 2, wherein said region is region 2, and an amino acid sequence GHSQGEI, is changed to GHSLGEI, GHSVGEI, or GHSIGEI.

12. The method of claim 2, wherein said region is region 3, and an amino acid sequence VRYASHS is changed to VRHAFHS, or VRHGFHS.

13. The method of claim 1, wherein said region is region 3, and an amino acid sequence His-X-Phe-His is changed to Tyr-X-Ser-His, Tyr-Ala-Ser-His, or Tyr-Gly-Ser-His.

14. The method of claim 1, wherein said region is region 3, and an amino acid sequence His-X-Phe-His is changed to Thr-Ala-Gly-His or Cys-Pro-Thr-His.

15. The method of claim 1, wherein said region is region 3, and an amino acid sequence Tyr-X-Ser-His is changed to His-X-Phe-His, His-Ala-Phe-His, or His-Gly-Phe-His.

16. The method of claim 1, wherein said region is region 3, and an amino acid sequence Tyr-X-Ser-His is changed to Thr-Ala-Gly-His or Cys-Pro-Thr-His.

17. The method of claim 1, wherein said region is region 3, and an amino acid sequence selected from the group consisting of His-X-Phe-His, His-Ala-Phe-His, Tyr-X-Ser-His, and Val-X-Ser-His, is changed to an amino acid sequence selected from the group consisting of Thr-Ala-Gly-His, Thr-Ala-Ser-His, Cys-Pro-Thr-His, Phe-Ala-Gly-His, and Val-Ala-Gly-His.

18. The method of claim 1, wherein said region is region 3, and an amino acid sequence selected from the group consisting of Thr-Ala-Gly-His, Thr-Ala-Ser-His, Cys-Pro-Thr-His, Phe-Ala-Gly-His, and Val-Ala-Gly-His, is changed to an amino acid sequence selected from the group consisting of His-X-Phe-His, His-Ala-Phe-His, Tyr-X-Ser-His, and Val-X-Ser-His.

19. A Streptomyces host cell that produces the compound of claim 3.

20. A Saccharopolyspora erythraea host cell that produces the compound of claim 3.
